(19) United States
(12) Patent Application Publication
(10) Pub. No.: US 2019/0160203 A1
(43) Pub. Date: May 30, 2019

(54) PREPARATION AND APPLICATIONS OF RGD CONJUGATED POLYSACCHARIDE BIOINKS WITH OR WITHOUT FIBRIN FOR 3D BIOPRINTING OF HUMAN SKIN WITH NOVEL PRINTING HEAD FOR USE AS MODEL FOR TESTING COSMETICS AND FOR TRANSPLANTATION

(71) Applicant: CELLINK AB, GOTHENBURG (SE)

(72) Inventor: Paul Gatenholm, Riner, VA (US)

(21) Appl. No.: 16/306,436

(22) PCT Filed: Jun. 3, 2017
(86) PCT No.: PCT/US17/35861

§ 371 Date: Nov. 30, 2018

Related U.S. Application Data
(60) Provisional application No. 62/345,597, filed on Jun. 3, 2016.

(51) Int. Cl. A61L 27/38; C12N 5/077; A61L 27/36; A61L 27/60; A61L 27/54; C12N 5/071

(52) U.S. Cl. CPC A61L 27/38; C12N 5/0656; A61L 27/3687; B33Y 10/00; A61L 27/54; C12N 5/0698; A61L 27/60

(57) ABSTRACT

The present invention relates to use of hydrogel based on RGD-conjugated alginate with and without addition of nanocellulose and/or fibrin as a novel bioink for 3D Bioprinting of human skin, particularly dermis. RGD-conjugated alginate provides adhesion sites for the human fibroblasts which result in cell adhesion and stretching which contribute to upregulation of genes producing Collagen I. In this invention, RGD-conjugated alginate is used as one of the components of the bioink for 3D bioprinting. Another innovation described herewith is use of coaxial needle when 3D bioprinting with alginate and RGD-modified alginate bioinks. A coaxial needle makes it possible to crosslink the bioink upon 3D bioprinting operation and thus achieve high printing fidelity which is required for high cell viability, proliferation and production of extracellular matrix. In this invention, the novel RGD-modified alginate bioink together with human fibroblasts is 3D bioprinted and the resulting construct shows high cell viability, high cell proliferation, high degree of stretching of fibroblasts and high productivity of Collagen I. The cell bioink construct biofabricated with this invention is ideal for testing cosmetics and active ingredients of skin care products particularly those used for skin regeneration. It is also ideal to be used as skin grafts for skin repair for patients with damaged or burned skin.

### BACKGROUND OF THE INVENTION

### Field of the Invention

The present invention relates to hydrogels based on polysaccharides, such as alginate and nanocellulose and particularly RGD conjugated alginate and RGD conjugated nanocellulose combined with fibrin for use as novel bioinks to be used with 3D Bioprinting technology and a combination of these novel bioinks with a coaxial printing needle. These novel bioinks are particularly suitable for 3D cell culturing of human fibroblasts and growing human skin. In this invention RGD-conjugated alginate is used in the formulation of the 3D Bioprinting bioink with non-conjugated alginate. The composition of the bioink is designed to provide optimal rheological properties which gives high printing fidelity. Nanocellulose is added to control rheological properties whereas fibrin is added to provide suitable environment for fibroblasts to proliferate and produce an extracellular matrix, preferably Collagen I. A critical aspect claimed by this invention is the presence of RGD peptide conjugated to alginate, which affects adhesion and spreading of human fibroblasts, as well as the presence of fibrin. The spreading of human fibroblasts activates the cells and results in upregulation of Collagen I production, which is a major component of the skin. Bioinks described herein were printed with and without a coaxial needle providing fast crosslinking upon bioprinting and giving optimal printing fidelity which resulted in high cell viability. Bioink described in this invention can be 3D bioprinted with or without human fibroblasts, but mixing and 3D bioprinting with human fibroblasts in the mode known as cell-laden hydrogel is preferred. Embodiments of this invention relate to human skin and particularly the dermis layer of the skin. Epidermis is the top layer of the skin and it consists of several types of cells such as keratinocytes, melanocytes and Langerhans cells. Keratinocytes are the most abundant cell type. Epidermis is much thinner than dermis which typically is 1-4 mm thick, depending on the location in the body. The invention describes how the bioink is mixed with cells, 3D bioprinted, and cultured to become a model for skin which can then be used for testing of cosmetics, skin care products and be used for transplantation. It can also be used for high throughput drug discovery, screening, and toxicity testing. Alternatively it can be directly implanted in a wound.

### Description of Related Art

Skin is the human body's largest organ. It is composed of two layers; epidermis, which is the outermost layer and consists mainly of keratinocytes, which, during the process called stratification, are converted into dense layer(s) of keratin which act as a barrier. The second layer, dermis, is mainly composed of dermal fibroblasts which are responsible for production of extracellular matrix. The major component of extracellular matrix of dermis is Collagen I. During the human aging process, the production of Collagen I is decreased and also connections between the Collagen I network and fibroblasts decreases. This results not only in damage to the skin, but also the presence of wrinkles. The cosmetic and skin care industry has been working to develop products containing active substances which can enhance proliferation of fibroblasts and increase production of Collagen I. New products were evaluated in Europe until 2013 mostly by testing the skin products through animal testing. Since 2013, a ban has been levied against animal testing of cosmetic products in Europe and the cosmetic industry is researching to develop other models of human skin.

Additionally, 11 million people worldwide suffer from burn injuries requiring medical intervention. 300,000 people die every year due to burn injuries. The burns, which are second, third or fourth degree, require urgent treatments. Currently, skin grafts are used to help these patients. Autologous skin grafts are preferred but the burned patients often lack the undamaged skin to be transplanted. Patients' own skin, which could be grown in a laboratory, would help to save the lives of thousands of patients.

3D Bioprinting is an emerging technology which enables biofabrication of tissue and organs. The technology is based on using 3D bioprinters, which comprise a robotic arm that dispenses liquid biomaterial and cells in a pattern determined by CAD file blue prints to control the motion of the 3D bioprinter. It is taught herein that 3D Bioprinting technology may be used for biofabrication of human skin since the different layers can be printed with various cell densities with high resolution. The outcome of the 3D Bioprinting process will depend on the bioinks being used. Bioinks have the role of providing suitable rheological properties during 3D Bioprinting, cell viability, and also acting as scaffolds during tissue development.

Human fibroblasts need to attach in order to actively produce extracellular matrix. In native environments, such attachment takes place by binding to fibronectin, which contains Arg-Gly-Asp (RGD) domains that interact with cells through integrins, which are transmembrane cell adhesion receptors. Alginates have been used for many years as biomaterials for cell encapsulation and have many biomedical applications.

Cells do not bind to pure alginate. Conjugation of a variety of peptides facilitate and promote cell attachment. Peptide-coupled alginates can be prepared using aqueous carbodiimide chemistry as described by J. A. Rowley, G. Madlambayan, D. J. Mooney, Alginate hydrogels as synthetic extracellular matrix materials, Biomaterials 20 (1999), 45-53. Examples of materials described in this innovation are NOVATACH G/M RGD (GRGDSP-coupled high G or high M alginate), NOVATACH G VAPG (VAPG-coupled high G alginate), NOVATACH M REDV (REDV-coupled high M alginate) produced by FMC Biopolymers, NovaMatrix, Norway.

### SUMMARY OF THE INVENTION

In this invention, a preparation of a new bioinks is described, such as bioinks composed of: RGD-modified alginate; fibrin with or without addition of nanocellulose or RGD-modified nanocellulose; and fibrin with addition of alginate. This invention also teaches using such bioinks for printing with human fibroblasts. RGD-modified alginate provides attachments sites for integrins at the surfaces of fibroblasts resulting in cell stretching. Cell stretching has been shown to upregulate production of Collagen I, which makes such 3D Bioprinted constructs preferable for use as a dermis model for testing active substances in cosmetics or skin care products, or for skin transplantation. This invention also describes using a coaxial needle to crosslink alginate during a 3D Bioprinting process. When dermis is developed the keratinocytes can be seeded or 3D Bioprinted on the top of such dermis layer while full skin is developing.

### BRIEF DESCRIPTION OF THE DRAWINGS

The accompanying drawings illustrate certain aspects of embodiments of the present invention, and should not be used to limit or define the invention. Together with the written description the drawings serve to explain certain principles of the invention.

FIG. 1 is a depiction of a 3D Bioprinter INKREDIBLE from CELLINK AB, Sweden printing dermis constructs.

FIG. 2 is a depiction of fibroblasts-laden bioink constructs with preferable printing fidelity.

FIG. 3 is a depiction illustrating cell viability in a printed construct with RGD-alginate.

FIG. 4 is a depiction showing cell morphology in printed constructs after 14 days culturing.

a) Unmodified bioink b) RGD-modified Alginate c) RGD-modified Alginate and addition of TGFBeta to medium

FIG. 5 is a depiction showing 3D Bioprinting using a coaxial needle and an illustration of a preferred needle arrangement.

### DETAILED DESCRIPTION OF VARIOUS EMBODIMENTS OF THE INVENTION

The present invention has been described with reference to particular embodiments having various features. It will be apparent to those skilled in the art that various modifications and variations can be made in the practice of the present invention without departing from the scope or spirit of the invention. One skilled in the art will recognize that these features may be used singularly or in any combination based on the requirements and specifications of a given application or design. Embodiments comprising various features may also consist of or consist essentially of those various features. Other embodiments of the invention will be apparent to those skilled in the art from consideration of the specification and practice of the invention. The description of the invention provided is merely exemplary in nature and, thus, variations that do not depart from the essence of the invention are intended to be within the scope of the invention.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Embodiments of the invention include RGD-modified alginate bioink products prepared by the methods described and include using the products in 3D Bioprinting operations.

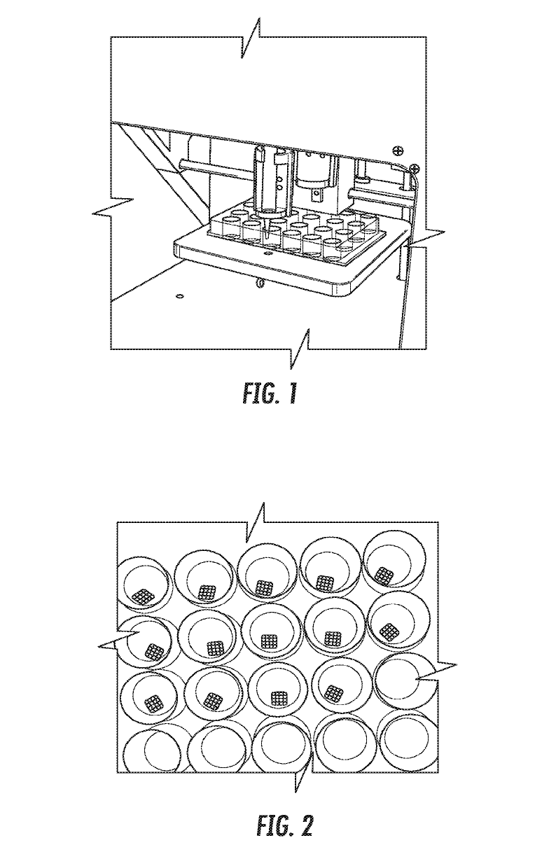

FIG. 1 is a depiction of a 3D Bioprinter INKREDIBLE from CELLINK AB, Sweden printing dermis constructs. These 3D printed dermis constructs may be cultured to become a model for skin which can then be used for testing of cosmetics, skin care products, and be used for transplantation. They can also be used for high throughput drug discovery, screening, and toxicity testing. Alternatively, they can be directly implanted in a wound.

FIG. 2 is a depiction of fibroblasts-laden bioink constructs with preferable printing fidelity. This is relevant for transporting nutrients and oxygen for the cells within the construct.

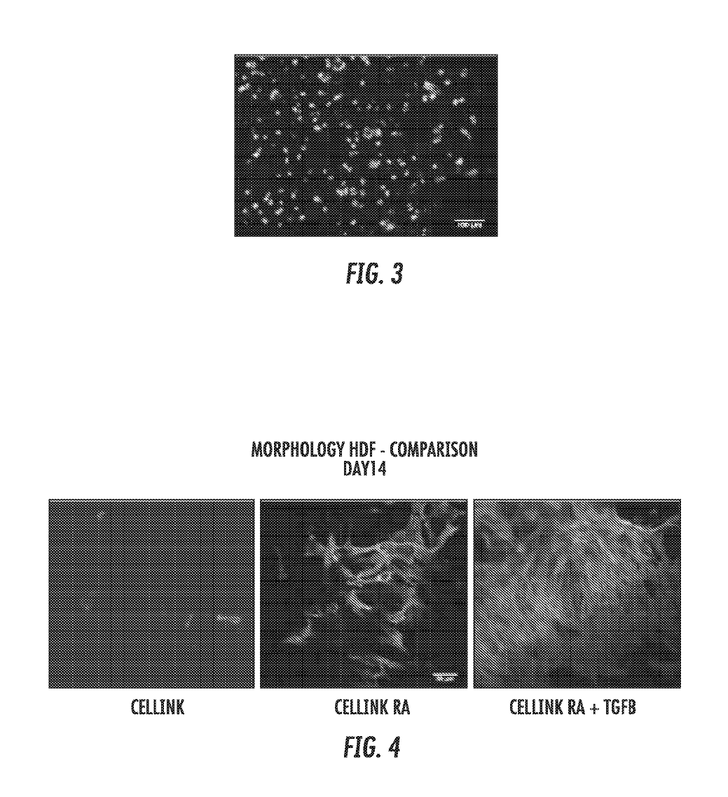

FIG. 3 is a depiction illustrating cell viability in a printed construct with RGD-alginate. Green spots represent cells which are alive, while red spots indicate dead cells. The cell viability is more than 70% in this depiction.

FIG. 4 is a depiction showing cell morphology in printed constructs after 14 days culturing. Green spots represent cytoskeleton and blue spots represent cell nuclei.

a) Unmodified bioink b) RGD-modified Alginate c) RGD-modified Alginate and addition of TGFBeta to medium

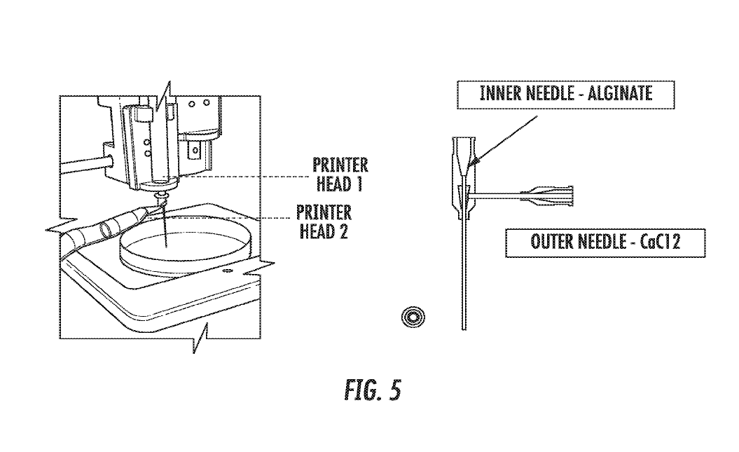

FIG. 5 is a depiction showing 3D Bioprinting using a coaxial needle and an illustration of a preferred needle arrangement. The coaxial needle provides faster crosslinking upon bioprinting and gives optimal printing fidelity, which, in a preferred embodiment, results in high cell viability.

To facilitate a better understanding of the present invention, the following examples of certain aspects of some embodiments are given. In no way should the following examples be read to limit, or define, the scope of the invention.

### Example 1

3D Bioprinting of Dermis-Like Model

Two different bioinks were prepared. The first bioink was composed of pure alginate with addition of nanocellulose to control rheological properties. The second bioink was prepared by combining RGD-modified alginate with nanocellulose to control rheological properties. Both bioinks had good printability. Six million primary human fibroblasts passage #3 were thawed and seeded into two 150 cm2 T-flasks. When the culture reached approximately 90% confluence, the cells were harvested using TrypLE and the flask was gently tapped to make the cells detach from the surface. The cells were counted (1.9 M cells/mL) with Tryphan-blue staining and the cell viability was calculated to ensure the cells were alive. The cells were then centrifuged and resuspended in medium and then seeded with 2,500 cells/cm2 into a T150 flask. The medium (DMEM, 1% GlutaMAX with 10% FBS and 1% Pen/Strep with phenol red) was changed three times per week. The cells were mixed with the bioinks to provide a final concentration of 5.2 million cells/ml and then carefully moved into the printer cartridge. Constructs were printed in a grid pattern in three layers with the dimensions of 6 mm×6 mm×1 mm (pressure: 24 kPa, feed rate: 10 mm/s) using the 3D-bioprinter INKREDIBLE from CELLINK AB, Sweden (see FIG. 1). After printing, the constructs were crosslinked with 100 mM CaCl₂ for 5 minutes. Thereafter, CaCl₂ was removed and the constructs rinsed with medium. The constructs were cultured statically for 14 days in incubator at 37° C.° and the medium was changed every third day. TGFBeta was added at a concentration of 5 ng/ml medium to some of the constructs. The constructs were analyzed for cell viability, morphology and collagen production after 14 days. Live/Dead staining was performed on 3 constructs from each bioink of the static culture on day 1, day 7, and day 14 using a LIVE/DEAD Cell Imaging Kit (R37601 Life Technologies). FIG. 3 shows good cell viability (more than 70%) for all printed constructs. On day 14, the static culture constructs were imaged using a confocal microscope. The FITC was used to visualize the cytoskeleton (green) and the DAPI was used to visualize the nuclei (blue) of the cells. Images were taken at 4×, 10×, and 20× magnification to analyze cell morphology. ImageJ was used to overlay images of the cytoskeletons and nuclei. FIG. 4 a) shows the morphology of fibroblasts in alginate bioink with addition of nanocellulose. The cells were round and not stretched. FIG. 4 b) shows fibroblasts in RGD-modified alginate bioink with addition of nanocellulose. The cells were stretched because they were able to attach to RGD peptides which were conjugated with alginate. FIG. 4 c) shows fibroblasts in RGD-modified alginate bioink with addition of nanocellulose cultured with additions of TGFBeta. The effects are noted as increased cell proliferation, and continued stretching. These effects were not seen for the cells printed with bioink which was not modified with RGD. The constructs were analyzed with PCR and the constructs with RGD-modified alginates showed upregulated genes for production of Collagen I.

### Example 2

### 3D Bioprinting of Full Skin with Nanocellulose, Alginate RGD and Fibrin Bioink

Bioinks were prepared using aseptic techniques from fibrinogen powder purchased from Sigma and hydrogels of 3% nanocellulose and 2.6% alginate conjugated with GRGDSP-peptides acquired from FMC Biopolymers, NovaMatrix. The inks were made by mixing the components into homogeneous hydrogels. For the inks containing fibrinogen, the nanocellulose and alginate hydrogels were first mixed and the fibrinogen was dissolved with 200 μL/10 mg fibrinogen tris Buffered Saline (TBS) acquired from Fisher BioReagents. By using a SpeedMixer™ DAC 150.1 FV-K, the fibrinogen was mixed in the hydrogel to a homogeneous hydrogel composed of fibrinogen, nanocellulose and alginate. Different amounts of fibrinogen were added to hydrogel bioinks ranging from 10 mg to 500 mg per 1 ml bioink. Two different types of cells were used; primary adult human dermal fibroblasts (aHDFs) and primary human epidermal keratinocytes (HEKs) both acquired from LifeLine® Cell Technology. They were both cultured according to protocol from the supplier in cell specific culture medium (FibroLife® and DermaLife®, respectively) before mixing with bioinks and bioprinting. A thrombin solution was prepared with 10 units/ml thrombin in 100 mM CaCl₂ to be able to crosslink the alginate and polymerize the fibrinogen simultaneously. The chosen construct model was a grid pattern in two layers. aHDFs were mixed in bionks in a concentration of 10 M cells/ml. Both lower and higher cell concentrations can be used. The printer used was a extrusion bioprinter (INKREDIBLE®, CELLINK®). The printing pressure for the fibrin based bioinks was between 12-23 kPa. After printing, the constructs were crosslinked and polymerized for 5 min using thrombin solution in 100 mM CaCl₂ before placing in culture medium. The constructs were then cultured in FibroLife® medium for two weeks. After two weeks HEK cells were seeded (30 M/ml medium) and samples were incubated for another two weeks. The samples for analysis were taken at 7 and 14 days and 28 days. After constructs were sliced and stained for pro-collagen and Masson's trichrome staining to get visualization of collagen production within the constructs. There was positive effect of the addition of fibrin on cell morphology and production of Collagen I.

### Example 3

### 3D Bioprinting of Constructs with Coaxial Needle

The constructs composed of fibroblasts laden RGD-alginate were prepared by 3D Bioprinting using a coaxial needle (see FIG. 5). The inner part of the needle was used to print with fibroblasts mixed with RGD-alginate whereas the outer part of the needle was used to eject 100 mmol solution of CaCl₂. Good printing fidelity was achieved using this method. In another experiment, fibroblasts laden RGD-alginate was combined with fibrinogen and 3D bioprinted using a coaxial needle. The inner part of the needle was used to print with fibroblasts mixed with RGD-alginate and fibrinogen whereas the outer part of the needle was used to eject thrombin solution dissolved in 100 mmol CaCl₂ solution. Good printing fidelity was achieved using this method.

One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1-52. (canceled)

53. A 3D-printable bioink comprising RGD-modified alginate and/or nanocellulose.

54. The bioink of claim 53, further comprising human fibroblasts.

55. The bioink of claim 54, further comprising fibrin.

56. The bioink of claim 54, wherein the nanocellulose is RGD-modified nanocellulose.

57. The bioink of claim 55, wherein the nanocellulose is RGD-modified nanocellulose.

58. The bioink of claim 53, further comprising fibrin.

59. The bioink of claim 53, further comprising human cells.

60. The bioink of claim 58, further comprising human cells.

61. The bioink of claim 58, wherein the nanocellulose is RGD-modified nanocellulose.

62. The bioink of claim 61, wherein the nanocellulose is RGD-modified nanocellulose.

63. A method of 3D bioprinting comprising:
bioprinting with one or more bioink comprising RGD-modified alginate and/or RGD-modified nanocellulose; and
forming a 3D bioprinted scaffold, living tissue and/or organ from the bioink.

64. The method of claim 63, wherein the scaffold is a dermis-like construct.

65. The method of claim 63, wherein the bioink does not comprise fibroblasts.

66. The method of claim 65, further comprising seeding fibroblasts on the 3D bioprinted scaffold.

67. The method of claim 63, wherein the bioink comprises fibroblasts.

68. The method of claim 63, wherein the bioink comprises human cells.

69. The method of claim 63, wherein the bioink does not comprise human cells.

70. The method of claim 63, wherein the scaffold, living tissue and/or organ is skin, cartilage, bone, an aorta, trachea, meniscus or ear.

71. The method of claim 63, wherein the bioprinting is performed using RGD-modified alginate with nanocellulose and/or RGD-modified nanocellulose.

72. The method of claim 71, wherein the bioprinting is performed using RGD-modified alginate with alginate and/or fibrin.
